(19) United States
(12) Patent Application Publication
(10) Pub. No.: US 2024/0237975 A1
(43) Pub. Date: Jul. 18, 2024

(54) CANCER DIAGNOSTIC DEVICE

(71) Applicants: PREVIEW MEDICAL INC., Longmont, CO (US); THE REGENTS OF THE UNIVERSITY OF COLORADO, A BODY CORPORATE, Denver, CO (US)

(72) Inventors: Sharon Lyn LAKE, Longmont, CO (US); Priya Niranjan WERAHERA, Aurora, CO (US)

(73) Assignees: PREVIEW MEDICAL INC., Longmont, CO (US); THE REGENTS OF THE UNIVERSITY OF COLORADO, A BODY CORPORATE, Denver, CO (US)

(21) Appl. No.: 18/559,136

(22) PCT Filed: May 5, 2022
(86) PCT No.: PCT/US2022/072124

§ 371 Date: Nov. 6, 2023

Related U.S. Application Data
(60) Provisional application No. 63/185,264, filed on May 6, 2021.

(51) Int. Cl. A61B 10/04; A61B 1/018; A61B 1/07; A61B 10/02; A61B 1/06; A61B 1/267

(52) U.S. Cl. CPC A61B 10/04; A61B 1/018; A61B 1/07; A61B 10/0266; A61B 1/0669; A61B 1/2676

(57) ABSTRACT

The present disclosure relates a system and method for tissue analysis, biopsy and/or treatment. In one example, a device is disclosed that includes a light generator and a probe in optical communication with the light generator. The probe includes an outer wall having a first end and a second end opposite the first end, a plurality of receiver fibers that extend along a length of the outer wall and are positioned between the first end and the second end, and a plurality of transmitter fibers that extend along a length of the outer wall, adjacent to the plurality of receiver fibers.

### CROSS REFERENCE TO RELATED APPLICATIONS

This application claims priority to U.S. Provisional Application No. 63/185,264 titled “Cancer Diagnostic Device,” filed on May 6, 2022, which is incorporated by reference in its entirety for all purposes.

### FIELD

This application generally relates to medical diagnostic, treatment, and biopsy devices and more specifically to optical spectroscopy devices with tissue classification, biopsy and/or treatment components.

### BACKGROUND

Lung cancer is the leading cause of cancer-related mortality in the US. An estimated 235,760 men and women will be diagnosed with lung cancer in 2021 and 131,880 will die from this disease in the US. Despite improvements in diagnosis, staging and treatment, the overall 5-year survival for lung cancer is still low between 10-20%, due to the very poor prognosis of many patients diagnosed at advanced stage. On the other hand, patients with early-stage lung cancer have a 5-year survival >70%. Hence, screening and detection of early lung cancer or premalignant lesions is crucial to improve survival. While the best defense against reducing mortality remains early diagnosis, some of these cancers are difficult to diagnose at early stages.

Cancer diagnosis requires histopathological confirmation of the disease by examining biopsy tissues. Transthoracic and bronchoscopy guided biopsies of solitary pulmonary nodules (SPN) are used to diagnose lung cancer. SPN is a round or oval spot/lesion in the lung typically visible with a chest x-ray or computerized tomography (CT) scan. A clinical challenge is the distinction of primary lung cancer from the perilesional lung parenchyma. Further, biopsy or tissue collection tools may be difficult to use depending on the location of the potential cancerous lesion. For example, many biopsy tools may require a rigid housing, which may prevent use in certain anatomical locations within the body. This leaves many cancers undetected until an advanced stage, reducing survival, and requiring more aggressive treatment options.

As a specific example, transthoracic needle aspiration is a method used to sample peripheral pulmonary nodules as it has a high diagnostic yield, e.g., 80% to 90%. However, this method also includes a 12% to 45% risk of pneumothorax with 2% to 15% requiring chest tube placement. Bronchoscopy can be safer than transthoracic needle aspiration, but typically provides a much lower diagnostic yield (e.g., 38.5% to 63.7%), even when utilizing advanced bronchoscope techniques, since around 80% of the nodules do not exhibit a bronchus side leading directly to the peripheral lung lesion. As such, the diagnostic yield drops from 70% with a bronchus side to 31% without it. Furthermore, there are currently no reliable ways to diagnose pre-cancerous lesions.

Precise in vivo localization of the targeted lesion and other areas remains critical. As such, there is a need in the art for an improved diagnostic and/or biopsy device and method that can be utilized across a variety of locations in the human body, including lungs, gastrointestinal tract, and the like.

### BRIEF SUMMARY

The present disclosure relates to a system and method for tissue analysis, biopsy, and/or treatment. In one example, a device is disclosed that includes a light generator and a probe in optical communication with the light generator. The probe includes a flexible outer wall having a first end and a second end opposite the first end, a plurality of receiver fibers having ends that are arranged to extend along a length of the outer wall and are positioned between the first end and the second end, and a plurality of transmitter fibers having ends that are arranged to extend along a length of the outer wall, adjacent to the plurality of receiver fibers.

In one example, the ends of the plurality of fibers may be arranged to extend annularly around a surface of the probe and may define annular sensing segments that extend around the surface of the probe.

In another example, the ends of the plurality of fibers may be arranged to extend longitudinally along a surface of the probe and may define longitudinal extensions of sensing segments.

In another example, a device is disclosed that may include a light source and a probe in optical communication with the light source. The probe may include a body, a first sensing segment positioned at a first location of the body and a second sensing segment positioned at a second location of the body, where the first location and the second location are located apart from a terminal end of the probe.

In yet another example, a device is disclosed including a probe having a flexible body defining a channel and a motor assembly coupled to the body and configured to move the body in precise movement intervals, a light source coupled to the probe, and a plurality of fibers positioned at least partially within the channel of the body. The plurality of fibers include one or more transmitting fibers configured to transmit light from the light source to one or more in vivo locations within a body and one or more receiving fibers extending through the body and configured to receive light from the one or more in vivo locations. The plurality of fibers are arranged to transmit and receive light through a sidewall of the flexible body. The device may also include a light sensor that translates light received from the one or more receiving fibers into digital data.

### BRIEF DESCRIPTION OF THE SEVERAL VIEWS OF THE DRAWINGS

FIG. 1A is a perspective view of a device for analyzing tissue within a body.

FIG. 1B is a simplified block diagram of a system including the device of FIG. 1A.

FIG. 2A is an enlarged view of a probe of the device of FIG. 1A illustrating a first example of a biopsy tool.

FIG. 2B is an enlarged view of the probe of the device of FIG. 1A illustrating a second example of a biopsy tool.

FIG. 3A is an enlarged end view of the probe of the device of FIG. 1A illustrating a third example of a biopsy tool.

FIG. 3B is an enlarged elevation view of the probe of FIG. 3A.

FIG. 4A is an enlarged view of the probe of the device of FIG. 1A illustrating a first example of a fiber arrangement.

FIG. 4B is a cross section view of the probe taken along line 4B-4B in FIG. 4A.

FIG. 5A is an enlarged view of the probe of the device of FIG. 1A illustrating a second example of a fiber arrangement.

FIG. 5B is a cross section view of the probe taken along line 5B-5B in FIG. 5A.

FIG. 6 is a perspective view of the probe of the device of FIG. 1A incorporated into a working channel of another device.

FIG. 7 is a flow chart illustrating a method of utilizing the system of FIG. 1B to analyze tissue.

### DETAILED DESCRIPTION

The present disclosure relates generally to devices and methods for tissue classification and/or tissue collection, as well as treatment. In one embodiment, a probe optically coupled to a light generator or light source is disclosed. The probe may include a housing or outer wall and a plurality of transmitting fibers that transmit light from the light generator to a tissue area (e.g., in vivo) and a plurality of receiving fibers that receive light reflections from the tissue area and transmit the received light to an analysis module, which may be incorporated in or separate to the light generator.

The transmitting and receiving fibers are positioned so that they emit light perpendicular or at another non-parallel angle to a length of the probe outer wall. For example, ends of the transmitting and receiving fibers may be oriented facing towards the probe outer wall. The fibers may bend along their length to gradually transition from extending parallel to a longitudinal axis of the probe and have a larger angular range than 90 degrees or the fibers may extend most of the length of the probe so as to be parallel with the longitudinal axis and at a sensing area may include a more severe bend, e.g., 90 degrees, to be oriented at an angle relative to the longitudinal axis. In some embodiments, the transmitting and receiving fibers may be clustered in groups to define segments with transmitting and receiving capabilities, e.g., sensing segments. The sensing segments may be defined in longitudinal lengths that extend along a length of the wall parallel to a center axis of the probe and/or may be defined annularly around a width of the probe wall, such as in a ring configuration.

In some embodiments, the fibers may be positioned between a first end and a second end of the probe, e.g., terminate at a location spaced apart from a terminal or distal end of the probe. In many embodiments, the fibers may include a bend or inflection such that a first portion of the fiber may extend longitudinally within the probe and parallel to a center axis of the probe and a second portion of the fiber may extend normal or at an angle to the extension.

The second portion can then be oriented to emit or receive light at locations (e.g., sensing locations) that are spaced from the terminal end of the probe.

In various implementations, the probe may also include a working channel configured to receive one or more tissue collection tools, e.g., biopsy tools, needles, brush collectors, or the like. Additionally or alternatively, the probe may also be configured to be received within a working channel of another device, e.g., a bronchoscopy device. In these embodiments, the probe can be used to supplement or enhance the features of the primary or main device. It should be noted that in many conventional bronchoscopes the external diameter is generally around 5-6 mm and is too large to examine beyond fourth to fifth order bronchi in adults. As such, the diameter for the working channel and/or probe may be selected to be sufficiently small to allow further examination.

In these embodiments, the fibers are positioned to collect data at various locations of the probe, rather than just at a terminal end, allowing more comprehensive scanning of a volume of tissue within a region of interest than conventional spectroscopy tools with only a single sensor with measurement localized to one location at a time and also lack volumetric assessment of tissue. The proposed probe can sense multiple locations along as well as around the probe. Further, in many instances, the probe may be flexible and of sufficiently small diameter to allow insertion into non-linear areas of a body, such as, into SPN in lungs located outside of the bronchial pathway in a minimally invasive manner.

The light emissions may be configured to both identify biomarkers in tissue and/or deliver treatment modalities to tissue (e.g., tissue characteristics). In some embodiments, the same fibers used for the spectroscopy may be used to deliver treatment. In other embodiments different fibers (either on the probe or in a separate device) may be used. For an example, tissue may undergo apoptosis/necrosis from treatment due to either thermal ablation from a laser or photothermal ablation using an exogenous fluorophore exited from probe fiber.

In some examples, sensitivity, specificity, positive predictive value (PPV), and negative predictive values (NPV) of white light video bronchoscopy (WLB) in detection of premalignant lesions were 26.5%, 63.9%, 34.4%, and 54.9%, respectively; the corresponding values for auto fluorescence imaging (AFI) were 52%, 79.6%, 64.6%, and 69.9% respectively, for narrow band imaging (NBI) were 66%, 84.6%, 75.4%, 77.7%, respectively, while the values for combination of NBI and AFI were 86.1%, 86.6%, 84.6%, and 88%, respectively. Additionally, biomarkers may be used as classifiers in auto fluorescence spectra of the lung tissue. Tryptophan, Collagen, and NaDH concentrations are subjected to change based on when tissue changes from benign, premalignant, and malignant status. These changes can be quantified in real-time using optical sensors adjunct to bronchoscopy devices enabling classification.

Elastic scattering and diffuse reflectance spectra of lung tissue can also be used to identify precancerous and cancerous lesions among SPNs. The light transmitted into the tissue will be scattered and absorbed by the tissue. After multiple scattering, some of the transmitted light will re-emerge through the bronchial surface into the air. This re-emergence is called diffuse reflection. The amount of diffuse reflection is determined by both scattering and absorption properties of the bronchial tissue. The stronger the absorption, the less the diffuse reflection; the stronger the scattering, the larger the diffuse reflection. Optical depth of penetration >1 cm can be achieved within the “therapeutic window (600-1000 nm)” where absorption due to oxy, deoxy hemoglobin (blood) and water is minimum. Diffuse reflectance spectroscopy in the range of 360-2400 nm demonstrated a sensitivity of 77.3% and specificity of 73.1% in differentiating between malignant and benign lung tissue and a sensitivity of 90.9% and specificity of 100% in differentiating malignant from necrotic lung tissue. Sensitivity and specificity can be further improved for diagnosis of lung cancer lesions by combining autofluorescence and diffuse reflectance spectra.

In various embodiments, the probe may be rotated and/or linearly moved during use to allow scanning (of the underlying tissue, which may be automated (e.g., motorized with an adjustable or hardwired stepping mechanism or other control), and to provide feedback on tissue characterization. Additionally, in instances where the probe is coupled to or includes a tissue collection tool, the device may capture tissue classified as suspicious (e.g., biopsy) for histopathological verification. In some embodiments, the probe may be used to diagnose SPNs outside of the bronchial pathways, e.g., a small gauge probe may be designed to penetrate bronchial pathway from the nearest point to an SPN.

In some examples, the movement of the probe and activation of the light generator is controlled or otherwise known, such as via a motor or visual markers, such as tick marks or fiducials. This allows the device to collect tissue characteristic information at known intervals along a path of movement, e.g., linear, rotational, or the like. The known movement information along with the tissue characteristics can generate a three-dimensional map of suspicious/not suspicious tissue. For example, a “tunnel” of tissue characteristics can be mapped by the probe, allowing identification of a width of a lesion or the like. As another example, the probe movement may be rotational, e.g., spinning around an axis while selectively capturing tissue characteristics, such as at every 10 degrees of rotation. Such correlated movement and spectroscopy readings allows a 360 degree mapping of tissue characteristics and if combined with known linear movement of the probe can further map suspicious versus not suspicious tissue locations within the body.

In many embodiments, the probe is flexible to allow navigation through non-linear areas of the body, e.g., the lungs. In some embodiments, a portion of the probe, such as the tip, may be configured to be stiffer than the remaining portions of the probe, to allow the probe to be pushed, driven, or otherwise navigated through the body. In some embodiments, the flexibility of the probe or portions thereof (e.g., the tip) is configured to be modified to switch between flexibility and increased stiffness, such as to biopsy elements. As a specific example, a tip portion of the probe may be formed of a phase shifting material, such as nickel titanium (nitinol), and an activation signal can be transmitted to the tip to allow it to transform from a flexible configuration to a stiffer configuration. Once transformed, the tip may be used to penetrate tissue walls and/or excise tissue.

In specific embodiments, the probe may be utilized to augment lung bronchoscopy devices to increase sensitivity and/or specificity of premalignant lesions diagnosis (Squamous metaplasia, mild, moderate, or severe dysplasia, and carcinoma in situ were regarded as histologically positive lesions), reduce false positive rates of diagnosing malignant lesions, and/or increase overall accuracy with a multisensory multi-scanning endoscopy device compatible with the working channel.

Generally, the devices and methods disclosed may be utilized to diagnose, biopsy multiple locations in a minimally invasive and/or automated manner within a body and/or treat tissue, including the lungs, intestinal tract, pancreas, and the like. As an example, locations that can be accessed with a scope (e.g., bronchoscope, endoscope, colonoscopy), can be accessed via the devices and methods described herein. However, other tools, such as robotic and advanced navigation platforms may be combined with the methods and devices described here to access the desired locations. These supplemental or additional tools may also be used to deliver treatment to tissue at desired locations either in combination or separate from the probe.

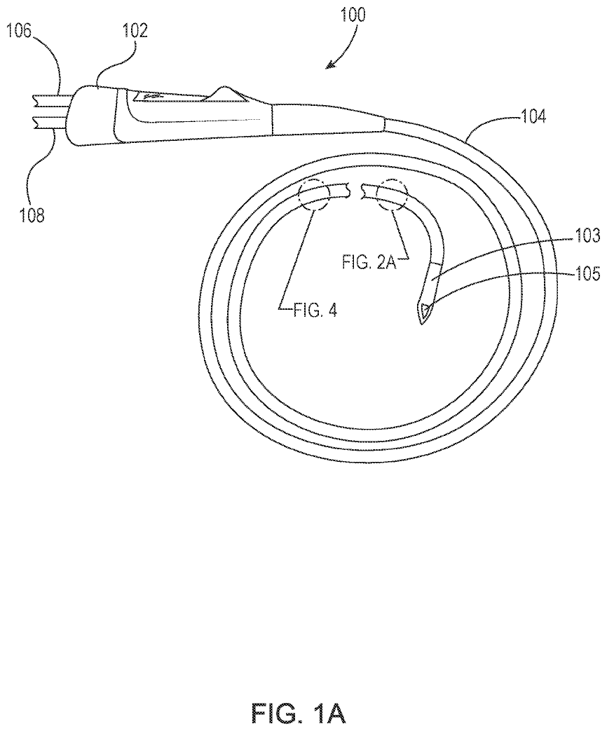

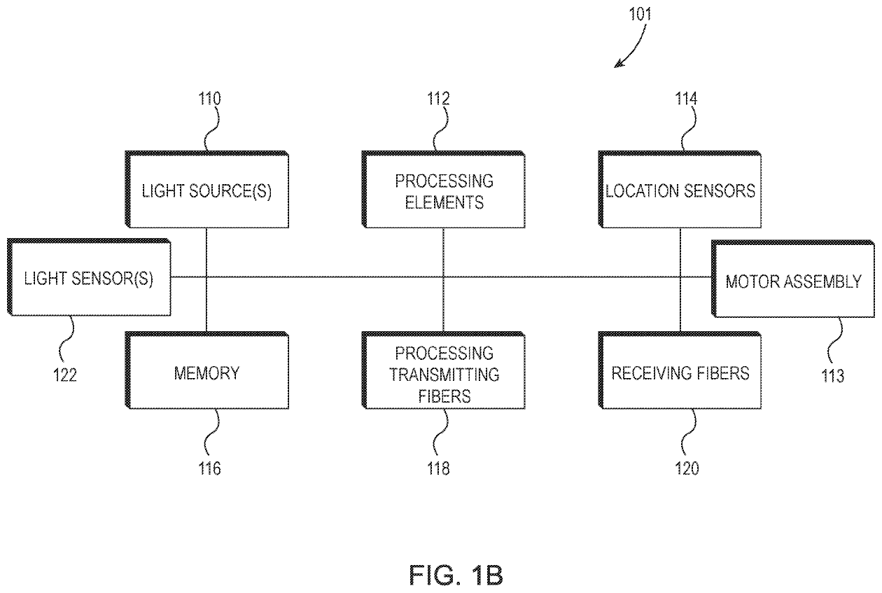

Turning to the figures, examples of the disclosure will be discussed in more detail. FIG. 1A is a device 100 including a flexible optical spectroscopy mechanism. FIG. 1B is a simplified block diagram of a system 101 including the device 100 or integrated to the device 100. The device 100 may include a handle 102 coupled to a probe 104. The handle 102 may also be coupled to a power source via power cable 106 and a light source 110 or light generator via light connector 108. The light connector 108 is optically coupled to the probe 104 as discussed in more detail below.

As shown in FIG. 1B, the system 101 may include one or more light sources 110 or light generators, which may be any type of lighting emitting device or element. Examples of the light sources 110 include organic light-emitting diodes, organic light-emitting diodes, laser diodes, gas lasers, solid-state lasers, fiber lasers, bulbs (halogen, tungsten, xenon, etc.), flash lamps, and the like. The light sources 110 are configured to generate desired wavelengths in particular spectral regions so as to generate radiation in a desired range. The light sources 110 may be arranged in an array and may generate different wavelengths as desired. In embodiments with multiple sources, the sources 110 may be combined spatially in a multiplexing system, free space, in the fibers, or guided wave and may be configured such that the light or radiation is delivered via evanescent near-field waves or fair field.

The location sensors 114 include components capable of detecting characteristics that may be used to help position and steer the probe 104 within the body. For example, the location sensors 114 may include an ultrasound sensor that can detect sound waves and display images of the internal anatomy of the body. As another example, the location sensors 114 may include light (e.g., cameras), sound, or other sensors that may be onboard the probe 104 that can be used to provide information to the processing elements 112 that may be useful in steering and/or locating the probe 104. As can be understood, the location sensors 114 may be incorporated or coupled to the device 100 and/or may be in communication with the device 100 as part of the system 101, e.g., a diagnostic sonography). For example, magnetic resonance imaging (MRI), micro-MRI, cameras with live video feed, and the like may be utilized to help determine location of the probe. In one example, the probe may include a camera coupled thereto, such as at a sensing segment or at a terminal end. In these instances, the camera or other location sensor 114 acts to provide visual information regarding the location of the probe 104 within the body of the probe. The camera may also be attached to the working tool or other device that may be used with the probe 104.

The system 101 may also include one or more processing elements 112, which may be both on-board or coupled to the handle 102 and/or separate therefrom (e.g., external device). The processing elements 112 may be any type of computing device or devise, such as, but not limited, processor, central processing unit, mobile processors, digital signal processors, microprocessors, micro controllers, computer chips, graphical processing unit, distributed or cloud based processing resources, a server, or the like. The processing elements 112 may be configured to receive and execute instructions, such as instructions stored on memory 116, including algorithms and machine learning models. In many embodiments, the processing elements 112 may be used to determine positioning of the probe 104, instruct activation of the light sources 110, analyze received reflections from the probe 104, activate the probe 104, and the like. Because the processing of any particular activity may be done by any of the processing elements 112, it should be noted that some activities may be processed by the handle 102, whereas others may be processed by other devices, separate, from, but in communication with the handle 102. In this manner, the form factor and cost of the handle 102 may be reduced, while maintaining the functional capabilities of the handle 102. To that end, the system 101 may include multiple processing elements 112 all of which may together or separately define the processor elements or processor. For example, there may be separate processing elements that control a motor assembly 113, but that may communicate with other processing elements, such as a central processing unit or the like.

Further, in some embodiments, the processing elements 112 may be configured to control one or more display outputs, such as one or more graphical user interfaces, etc., as well as execute or cause to be executed one or more analysis algorithms.

With reference to FIG. 1B, the system 101 may also include a motor assembly 113. The motor assembly 113 assists in driving or moving the probe 104 within the body. For example, the motor 113 may be configured to convert electrical signals into mechanical movement (angular, rotational and/or linear). In many embodiments, the motor assembly 113 is controlled by the processing element 112 allowing precise movement of the probe 104 and correlating activation of the light generation and hence light reflection capture and tissue characteristic identification at known intervals or locations. Such correlation between movement and spectroscopy readings assist in mapping an area of suspicious tissue. In embodiments where the motor assembly 113 may not be included, the handle 102 or other portion of the device 100 may include visual markers to provide a visual output of the length of extension of the probe 104 and the visual markers may be used to allow the user to activate the light generator to analyze the tissue at known intervals. In one example, the probe 104 may be configured to move at 1 mm intervals or steps while collecting and analyzing data.

The system 101 may also include on or more memory components 116. The memory 116 may be any type of component configured to store data, such as, but not limited to, read only memory, random access memory, solid state drive, secure digital cards, or the like. The system 101 may include any number of memory components as may be desired. The memory 116 may be in communication, directly or indirectly, with the processing elements 112, and may store executable instructions for execution by the processing elements 112, such as executable instructions for executing one or more methods as disclosed herein.

The system 101 may include one or more light sensors 122 configured to receive light and convert the light into a signal. For example, a spectrometer can convert light into an electrical signal where each pixel indicate amplitude/intensity and wavelength of the light signal. For example, the light sensor 122 may be configured to receive optical signals from receiving fibers 120 and convert the received light or information into a digital signal. The light sensors 122 or light detectors may be any type of light capturing or sensing device, including charged-coupled devices, image sensors, or the like. The light sensors 122 may act as an electromagnetic receiver. The light sensors 122 or other components of the system 101 (e.g., processing elements) may discriminate the received light or electromagnetic radio into n wavelength intervals of the electromagnetic spectrum. For example, the output of the receiver for a single source input may be n (integrated) intensity values, the values representation an integral of intensity values over some interval of the wavelength spectrum. The discrimination of the electromagnetic radiation into n integrated intensity values may be accomplished in a number of manners, including, but not limited to, photodetectors with absorptive filters, spectrometers, and wavelength selected organic photodetectors. Examples of analyzing the received light are discussed in more detail below.

In instances where multiple light sources are used, responses from the tissue (e.g., scattering and reflectance of emitted light) may be correlated to individual light sources through time-division multiplexing, space-division multiplexing, or the like. For example, a first light source may be activated and the tissue response captured and then a second light source may be activated and the response captured, etc.

The transmitting fibers 118 and receiving fibers 120 are optically transmissive fibers (e.g., fiber optic cables) or other components that can receive and direct light or radiation between different locations. For example, the transmitting fibers 118 may be configured to receive light from the light source 110 and direct the light to a location within the body (as determined by the probe 104), and the receiving fibers 120 may be configured to receive light reflections as reflected from within the body and return the light to the processing elements 112 and/or light sensors 122. For example, the receiving fibers 120 may collect diffuse reflectance and/or fluoresce emissions from tissue under excitation generated by the transmitting fibers 118. It should be noted that although the transmitting fibers 118 and receiving fibers 120 may include groups or sensing segments, e.g., more than one, fibers, in other embodiments, depending on the tissue to be analyzed, optical transmissivity, and the like, there may be a single fiber for the transmitter fibers 118 and/or receiving fibers 120. Similarly, although the transmitting fibers 118 and receiving fibers 120 have been discussed as separate fibers, in other embodiments, the same fibers that transmit energy may receive energy as well. The fibers 118, 120 are positioned along one or more sections or lengths of the probe 104 between the terminal ends (e.g., distal and proximal ends) of the probe 104. Examples of the positioning of the fibers 118, 120 is discussed in more detail below.

With reference again to FIG. 1A, the handle 102 is generally configured to allow a user, such as a doctor, treating physician, or the like, to guide and activate functionality of the device 100. For example, the handle 102 may include one or more control buttons that help direct the probe 104, activate the motor assembly 113, activate the light source, activate a biopsy mechanism, or the like. It should be noted that while certain features may be controlled via the handle 102, these features and/or additional features may be activated or controlled from another device, such as a workstation, computer, or the like. Additionally, robotic surgical devices may be used to help guide and activate the probe 104. In these examples, the handle may be omitted and the probe may be integrated with or coupled to the robotic surgical device. In these instances the motor assembly 113 may be integrated with the robotic device. As such, the discussion any particular location for a control feature is meant as illustrative only. In many instances, however, the handle 102 may be configured to have form-factor and shape that allows the handle 102 to be grasped and held in the hand of a user, the easy to grip shape of the handle 102 may allow a user to more readily steer the probe 104 via manipulation of the handle 102. As noted above, the handle 102 is configured to allow precise and known movement of the probe 104, such as via motor control of the motor assembly 113 or via manual control with visual identifiers.

The probe 104 is used to guide the fibers 118, 120 into the body and to the desired tissue location. In some embodiments, the probe 104 may also be used to guide a biopsy tool, which may be separate from or integrated with the probe 104, to the tissue location. Further, the probe 104 may be used along with a separate biopsy tool (see FIG. 6). In many embodiments, the probe 104 may be a flexible member, but sufficiently rigid to support and steer the fibers 118, 120 through the body. For example, the probe 104 may include shape memory alloys with varying stiffness that can be activated between different phases and different rigidity values via electrical current or heat, e.g., nitinol. Additionally, in some instances, the probe 104 may be formed with one or more linked components, forming a linkage, that allows the probe to be flexible, but retain rigidity between the links. The probe 104 may be hollow or otherwise define a working channel that may allow tools to pass therethrough. For example, the probe 104 may define a working channel for biopsy tools to pass through, such that the probe 104 can assist in delivering the tools to the tissue location.

The probe 104 may be steered or directed by a user into the body. For example, a doctor may utilize the handle 102 to manipulate the probe 104 to direct the probe through orifices within the body to the desired tissue location, e.g., lungs, prostrate, etc. It should be noted that the probe 104 is configured to be minimally invasive when positioned within the body, e.g., can be inserted under local or general anesthesia. As noted, the steering of the probe 104 may be via the motor assembly 113, which selectively drives the probe 104, extending the length of the probe 104 as it is driven further into the body. The motor assembly 113 may drive the probe 104 both in a linear fashion and/or a rotational or annular manner. For example, the motor assembly 113 may rotate the probe about an axis or may linearly push the probe 104 along a line.

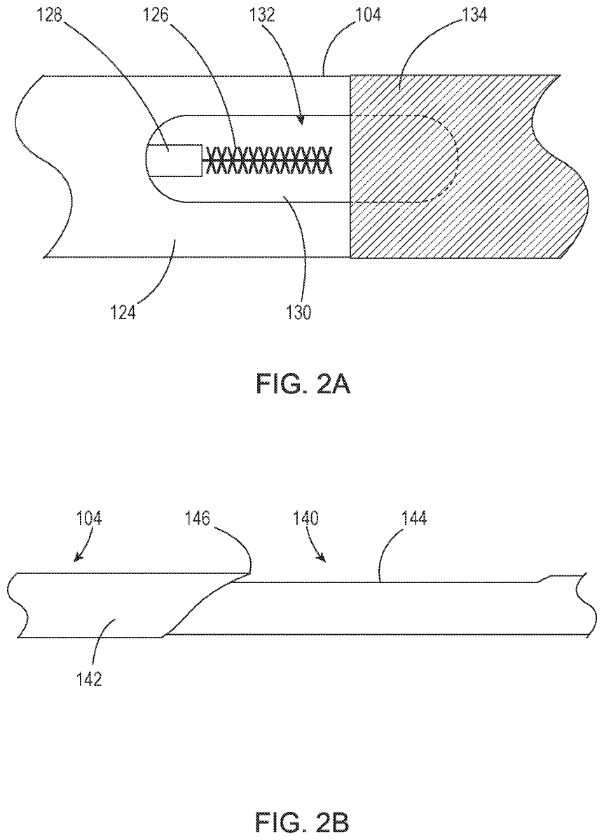

The probe 104 may include a tip 103 that forms a terminal end of the device. In some embodiments, the tip 103 may include a biopsying element, such as a pointed end or needle and optionally may include a compartment 105 or other cavity to receive tissue samples therein. It should be noted that while in FIG. 1A, the compartment 105 is shown as being at or adjacent to the terminal end of the probe 104, in other examples (see FIGS. 2A and 2B), the compartments for tissue collection may be located at other positions along the length of the probe 104. The location may depend on the type of biopsying element (if included) and the area to be biopsied.

For example, in FIG. 1A, the tip 103 may be configured as a hollow or partially hollow needle that can be inserted into a tissue to excise cells into the compartment 105, but other removal tools may be used that may excise and deposit cells in a different manner. Further, it should be noted that in some embodiments, the probe 104 may include a sheath or cover that extends over the pointed end of the tip 103 during navigation, e.g., before the probe 104 reaches a desired area for biopsying.

In other examples, the tip 103, which may form a terminal end of a body of the probe 104 (e.g., form an end wall or end tip and be coupled to a sidewall of the probe 104) may include another variation of a cutting element, such as a blade, scoop, or other cutting and collection configuration. In some embodiments, the terminal end of the probe 104 may be rounded or may not include a cutting element, and rather the cutting element may be deployed or actuated once the probe 104 is at a desired location or may be deployed to penetrate tissue to reach a desired location for spectroscopic analysis.

FIG. 2A is an enlarged view of an embodiment of the device 100. In this example, the probe 104 may include a body, such as a sheath or wall 124 that defines an interior cavity 132, such as a working channel, with an access aperture 130 defined within the wall 124 that provides access to the interior cavity 132. The wall 124 may define a sidewall of the probe 104. The body may be flexible to allow bending and curvature, while defining a channel therethrough. A cover 134 may be coupled to the wall 124 and configured to be selectively positioned to open and close, e.g., cover/uncover, the access aperture 130. The cover 134 may be actuated via an electrical signal, a manual activation, or the like.

A biopsy device 126, such as a brush collection tool, is housed at least partially within the interior cavity 132. The biopsy device 126 may be supported on the wall 124, such as via a mount, or may be otherwise coupled to the probe 104. In one example, the biopsy device 126 may be connected by a spring or flexible connection at the mount 128. In this manner, the biopsy device 126 is selectively actuated to be positioned radially outward or otherwise extend past the outer wall 124 of the probe 104 so as to be able to access tissue. In other words, the biopsy device 126 may be sheathed or housed to be protected during navigation to a particular location and then may be deployed to excise tissue or puncture tissue at a desired location.

For example, while the probe 104 or body 124 is being navigated to the desired tissue location, the cover 134 is positioned over the access aperture 130, concealing the biopsy device 126. Once the probe 104 is in the desired position, and optionally a tissue is identified as suspicious using the fibers 118, 120 described herein, the cover 134 is actuated to transition from the closed to the open position (e.g., deployed). Once opened, the cover 134 may no longer press against a spring within the mount 128, i.e., the cover 134 exerts a force opposite of the spring bias to retain the biopsy device 126 in the concealed or interior position within the interior cavity 132. With the cover 134 removed, the spring force causes the biopsy device 126 to extend outwards from the cavity 132. The user can then direct the probe 104 to brush against or engage the surface of the tissue, causing cells from the tissue to collect on the biopsy device 126, e.g., the biopsy device may include stiff fibers that scrape tissue cells off of the tissue location. After the biopsy is completed, optionally the user may actuate the cover 134, to close the cover 134. The cover 134 may be configured to slide over the biopsy device 126, acting against the spring force, causing the biopsy device 126 to collapse back into the interior cavity 132. The probe 104 can then be removed from the body.

FIG. 2B is an enlarged view of another example of the probe 104 with a different example of a biopsy device 140. In this example, an outer wall 142 or body of the probe 104 may also form a sheath or cover that may selectively cover/uncover an edge 144 or cutting implement. In one example, the cover 142 may include a pointed element 146 that may also act to slice or remove tissue cells when activated. In another example, the edge 144 may be manipulated by the user to scrape against the tissue to remove cells and the cover 142 may be activated to conceal or enclose the cells within the probe 104 after activation. For example, the edge 144 may be rotated against the tissue to excise cells. In other examples, the cover 142 may be omitted and the probe 104 may simply include an edge 144 or scraping side that can be selectively engaged with the tissue.

In other embodiments, the biopsy device may be configured as another blade configuration that allows removal of cells via rotational and/or linear motion. As another example, the biopsy device may be in the form of tongs or the like, depending on the area to be biopsied. As such, the discussion of any particular example of a biopsying device is meant as illustrative only. To that end, in some embodiments, the system 101 may include a vacuum or pump that may further assist in the collection and removal of cells. The vacuum may be selectively activated as the biopsy device is deployed to help direct and force cells into a collection chamber within the probe 104.

In operation, the user may direct the probe 104 to a desired location within the body. Optionally, a tissue location for biopsy may be determined by analyzing the reflectance via the fibers 118, 120. Once a tissue for sampling is determined, the user may manipulate the probe 104 to expose the edge 144 and then may position the edge 144 against the tissue. In embodiments where the cover 142 may assist in removing the cells, the cover 142 may be actuated to collect the cells. In other examples, the edge 144 may be moved against the tissue and sufficient sharp or designed to remove cells. Once the cells are removed, the cover 142 may be activated to enclose the cells and the edge 144, and the probe 104 may be removed from the body.

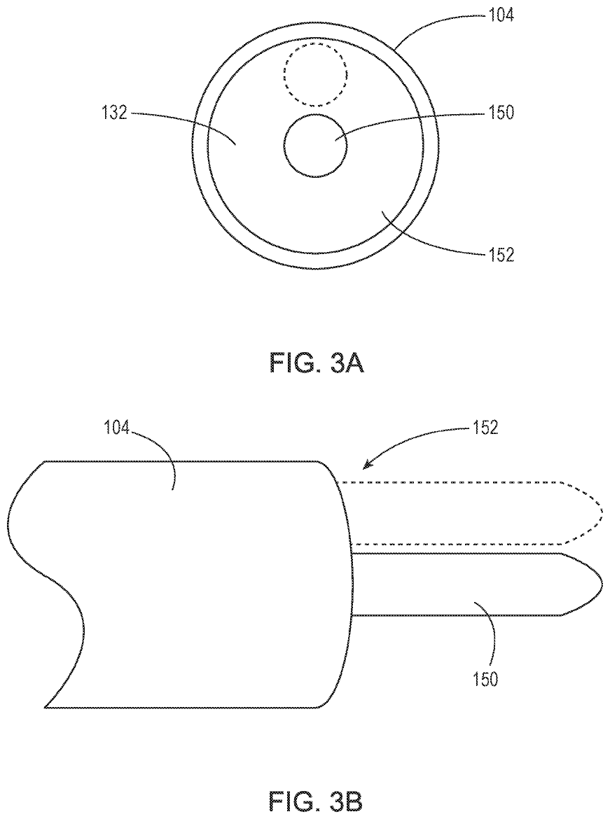

In some examples, the biopsy device or tool may be configured to extend from a terminal end of the probe 104. FIGS. 3A and 3B illustrate examples of a biopsy tool 150 that extends from a terminal end 152 of the probe 104. As shown, the biopsy tool 150, which may be a needle, or other similarly structured collection tool, can be positioned within the interior cavity 132 of the probe 104 and be configured to selectively extend past the terminal end 152 of the probe 104 as desired. Additionally, while shown as being positioned concentrically or in a central area of the interior cavity 132, the biopsy tool 150 may be otherwise positioned, such as off-centered or the like.

In use, the user may navigate the probe 104 to a desired location within the body, and once positioned, the user may activate the biopsy tool 150, causing it to extend from a retracted position to an extended position (or enclosed to exposed position). The user may then engage the biopsy tool 150 with tissue, to remove cells. Optionally, the biopsy tool may be retracted back into the interior cavity 132 of the probe 104 to be easily removed from the body. It should be noted that in some embodiments, the probe 104 may include a first navigational cutting element, e.g., the tip 103, that helps to cut through tissue to get to a desired biopsying location, such as within an SPN, and once positioned, a second biopsying tool, such as biopsying tool 150, is deployed and actuated. In these embodiments, the tip 103 may be configured to not collect cells, but rather simply puncture or otherwise define paths through tissue to other areas, such that cells are only collected at the desired location, rather than along the navigational path to the location.

Examples of biopsy tools that may be incorporated into the probe 104 can be found in U.S. Pat. Nos. 8,406,858 and 9,814,449, which are incorporated by reference herein for all purposes. In various embodiments, the biopsy tool 150 may be configured to both be fixed to the probe 104 and move therewith and/or may be configured to move separately from the probe 104. For example the biopsy tool 150 may be sheathed within the probe 104 during navigation to a biopsy location and move with the probe 104 to the location, then once at the desired location, the biopsy tool 150 may be deployed and moved separately from the probe 104, e.g., actuated and moved separately from the probe 104 to excise tissue and then retract back to the retracted position.

As mentioned, in many embodiments, the system 101 is configured to analyze tissue to determine biomorphometric properties and/or tissue characteristics that may be indicative of health of the tissue. Such embodiments may rely on optical spectroscopy, where light is directed towards tissue and reflections from the tissue are captured and analyzed. Light (photons) penetrate only few hundreds of microns into the tissue due to absorption from blood and water. In these instances, the light needs to be directed at the tissue from a close distance in order to ensure that a large percentage of the light reaches the tissue and reflectance waves can be captured. Thus, analysis of spectra provides biomorphometric properties of tissue immediately adjacent to the probe. Conventional spectroscopy devices may not be sufficiently flexible to allow navigation to certain areas of the body and/or are configured to emit light from just a terminal end, which requires a high precision of accuracy in aligning the probe against a tissue area or location. In many examples described herein, the probe 104 is flexible and includes extended lengths of exposure areas (e.g., fiber sections or sensing segments) to increase the surface area of the probe 104 that can be used to deliver and receive light. This helps to increase the precision in navigating the probe 104 to a desired area, as well can increase sensitivity of the probe 104 because more light can be delivered and collected than conventional tools. For example, the sensing segments may be positioned along the body of the probe 104, such that light can be emitted and received along the length of the probe, rather than just a terminal end, which can increase the surface area of exposure both for the tissue and the light, as well as reduce the sensitivity of the location steering required to analyze tissue.

In a specific implementation, such as in the diagnosis of lung tissue, the probe 104 may be configured to navigate outside of the bronchial pathways to reach SPNs, e.g., is flexible and configured to cut through or otherwise penetrate tissue once outside of the bronchial pathway.

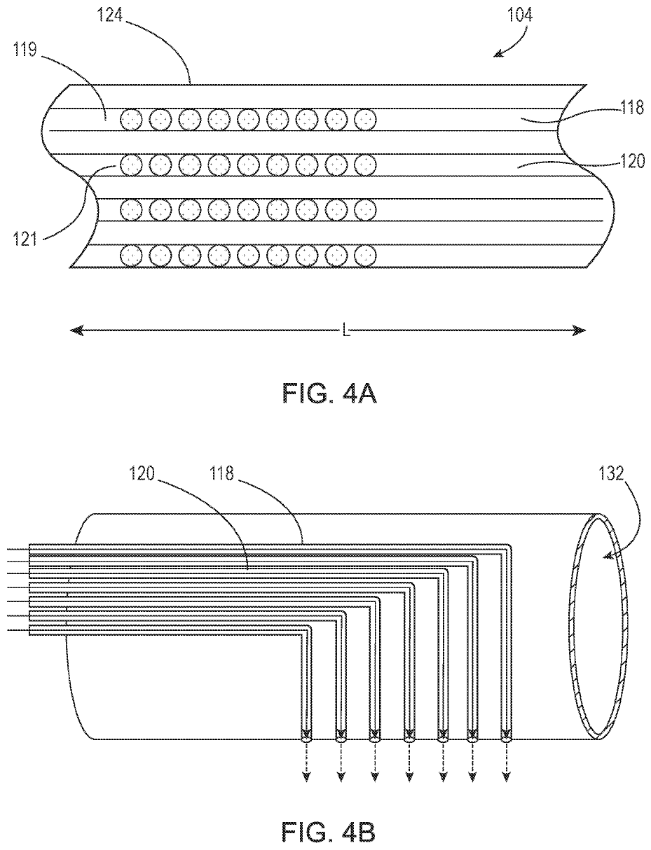

FIGS. 4A and 4B illustrate one example of the extended surface area for light collection/delivery of the probe 104. In this example, the probe 104 may include fibers 118, 120 that extend along a longitudinal length L of the probe 104 outer wall 124 or body. For example, the fibers may extend within a channel defined by the body. The fibers 118, 120 may be positioned within or behind the wall 124 or may be positioned on an outer surface of the wall 124. In embodiments where the fibers 118, 120 are embedded into the wall 124 or positioned behind the wall 124, the wall 124 may be at least partially if not fully transparent to allow light to extended therethrough or may include sections of transparent or partially transparent sections (e.g., transparent material or apertures). The wall 124 may act to shield and protect the fibers 118, 120 during navigation within the body to prevent damage to the fibers 118, 120. However, in many embodiments, the fibers 118, 120 may be exposed to increase sensitivity of the probe 104 and optionally allow the fibers 118, 120 to be directly positioned against the tissue to be analyzed. For example, the body may include apertures or windows that allow exposure of the fibers or ends thereof.

In this example, the fibers 118, 120 may be grouped in sensing segments 119, 121 that may include bunches of fibers arranged in longitudinal arrays that extend parallel to the length of the probe 104. The groups of sensing segments 119, 121 may be arranged to be parallel to one another and spaced apart from one another, both along the length and width of the probe 104. Adjacent segments 119, 121 may be aligned or may be misaligned. For example, a first sensing segment 119 may be positioned at a first length location of the probe 104 and a second sensing segment 121 may be positioned at a second length location that does not overlap with or partially overlaps with the second location, e.g., the sensing segments 119, 121 may be “stepped” along the length of the probe 104. In some embodiments, adjacent sensing segments 119, 121 may include different types of fibers, e.g., sensing segment 119 may include transmitting fibers 118 and sensing segment 121 may include receiving fibers 120. In other examples, each sensing segment 119, 121 may include different types of fibers 118, 120 which may be arranged in pairs or groups of two.

The fibers 118, 120 may be positioned such that the transmitting fibers 118 are positioned adjacent and parallel to the receiving fibers 120. In one example, the fibers 118, 120 may alternate, e.g., transmitting fibers may be positioned between receiving fibers. Additionally or alternatively, the fibers 118, 120 may extend around the entire outer surface or wall 124 of the probe, e.g., around the circumference. In some examples, the fibers 118, 120 may be positioned so as to be directly adjacent to one another, but in other examples, (as shown in FIGS. 4A and 4B), the fibers 118, 120 may be spaced apart from one another. The spacing or lack thereof may be determined by the characteristics of the fibers, desired light deliver/reception, tissue type, probe circumference, or the like. Relatedly, the fibers 118, 120 may extend along the entire longitudinal length L of the probe 104 or just a portion thereof. In the later example, the fibers 118, 120 may be positioned or exposed along selectively lengths, such as near a terminal end of the probe 104. In this manner, the fibers 118, 120 may be coupled to the light sources 110 by extending internally within the interior cavity 132 of the probe 104 until the desired exposure area or coupling to additional internal optical fibers that may be positioned within the probe 104. Diffuse reflectance spectra typically include single and multiple scattering events. Multiple scattering events is a function of the separation between source and read fibers. Consequently, diffuse reflectance spectra of tissue become a function of the separation between source fiber and read fiber (as described in more detail below).

In some embodiments, the fibers 118, 120 may include a bend or inflection to allow the fibers 118, 120 to extend parallel to the probe 104 body but bend at a respective location, e.g., sensing segment 119, 121, such that the terminal end of the fibers (e.g., emitting or receiving face) is oriented to be perpendicular to the length of the probe 104. This orientation allows the fibers to increase the light capturing or light emitting area to be maximized. For example, with reference to FIG. 4B, the fibers 118, 120 may each include a 90 degree or larger bend to orient the fiber ends to face outwards from a center axis of the probe 104. The bend may be arranged to reduce losses within light reflection, e.g., may be larger than a 90 degree bend to help maximize transmission, but still allow orientation of the fiber ends to be perpendicular or angled at another orientation relative to a longitudinal axis of the probe 104. In this manner, the bend may occur gradually along a length of the fibers 118, 120 rather than at a discrete location, e.g., a 90 degree bend.

In various embodiments, the terminal ends of the fibers 118, 120 may be configured to enhance transmission and reception characteristics. For example, the ends or exposed portions of the fibers 118, 120 may be polished and terminated such that light may be transmitted and received parallel to the fiber axis at the terminal end, rather than being diffused at the end.

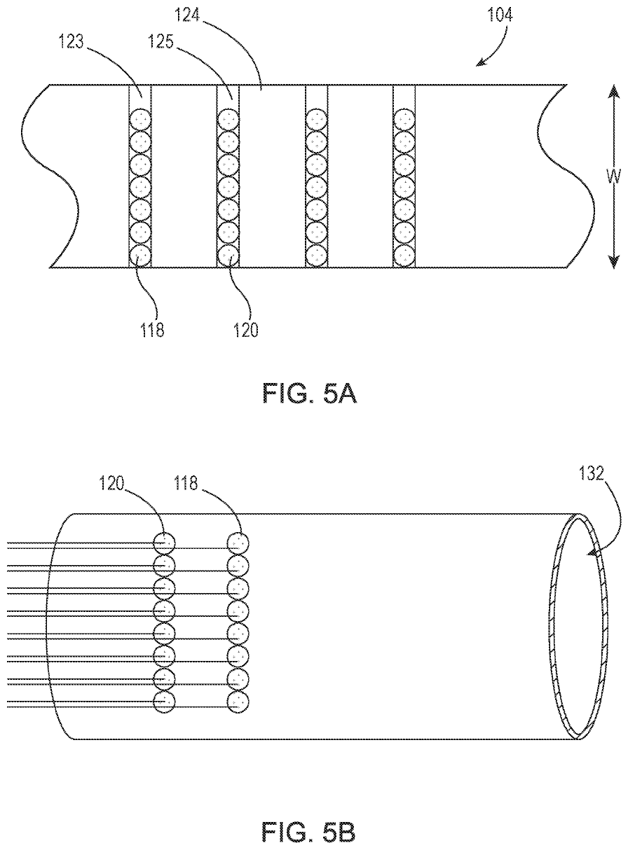

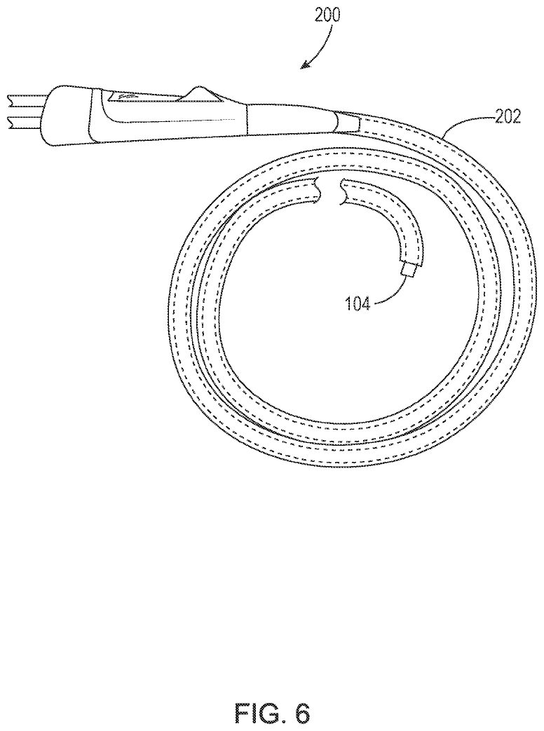

FIGS. 5A and 5B illustrate another example of the probe 104 with a different arrangement of fibers 118, 120, but also configured to increase the surface area of light transmissivity. In this example, the fibers 118, 120 may extend annularly around the outer wall 124 of the probe 104. In other words, the fibers 118, 120 may wrap around an outer circumference of the probe 104, and extend in a width W direction, rather than extend along their length. The fibers 118, 120 may wrap around the entire circumference or partially around the circumference. Specifically, the fibers 118, 120 may be arranged such that sensing segments 123, 125 may be defined as annular parallel rings that are spaced apart from one another along the length of the probe 104. For example, the sensing segments define sensing rings along various locations along the length of the probe 104. The sensing segments 123, 125 may include fibers at locations around the outer perimeter of the probe 104. As with the example of FIGS. 4A and 4B, the sensing segments may include a single type of fiber with alternating rings including different types of fibers, e.g., segment 123 may include transmitting fibers 118 and segment 125 may include receiving fibers 120. Alternatively, the sensing segments 123, 125 may include both types of fibers 118, 120, which may be arranged in pairs or other combinations.

As with the example in FIGS. 4A and 4B, in this example, the fibers 118, 120 may alternate, such that the layout may include transmitting fibers adjacent to receiving fibers, with the fibers 118, 120 being positioned directly adjacent one another or positioned spaced apart from one another. There may be groupings of fibers 118, 120 along the length of the probe 104, for example, every few inches or millimeters the probe 104 may include couplings of fibers 118, 120, e.g., bands of fibers, that extend around the circumference. In various examples, the fibers 118, 120 may also be exposed on the outer wall 124 of the probe 104 or embedded or concealed by the wall 124, where the wall 124 is at least partially transparent to allow light to be transmitted and received therethrough. The fibers 118, 120 may be configured to include a portion that extends within the interior cavity 132 of the probe and may extend along a length of the probe 104 to couple to the light sources 110. For example the fibers 118, 120 may have a bend or otherwise change angle or direction to extend longitudinally along a portion of the probe 104 to couple to the light source 110. See, for example, FIG. 5B illustrating the fibers 118, 120 having a bend or inflection such that the transmitting or receiving end of the fibers 118, 120 is perpendicular to a longitudinal axis of the probe 104.

With the examples of the fiber orientations illustrated in FIGS. 4A-5B, the probe 104 may have light detecting and emitting capabilities along larger surface areas and spaced apart from a terminal end. It should be noted that each of the fibers can act as a source or transmitting fiber, a detector or receiving fiber, or have dual functionality (e.g., both act to transmit and receive light). Fibers may also be configured to only receive or detect or to be placed in an optimal manner for certain wavelengths or properties of light, for example fibers may be configured to only collect data relating to diffuse reflectance spectra (which may provide more information regarding tissue underneath epithelial cell layers), single scattering spectra, or Rayleigh scattering. The various fiber configurations allow the probe 104 to analyze tissue samples more readily as it is navigated to a particular location, e.g., the light sources 110 can be activated during navigation or at waypoints to receive data regarding tissue health at different locations. Further, the positioning allows the probe 104 to be placed at a general area of interest, rather than a precise location and still be sufficient to collect data regarding the precise location. For example, as there may be multiple sensing segments along the length of the probe 104, the probe can be configured to analyze tissue against any of the sensing segments individually, which may increase the surface area of the tissue to be analyzed as well as reduce the sensitivity required to align the probe against a specific point of tissue as more tissue can be analyzed at a time.

FIG. 6 illustrates an example of a primary device 200 that may integrate with or couple to the probe 104. In this example, the probe 104 may be threaded through a working channel 202 of a primary device 200. As an example, the probe 104 may be utilized with a bronchoscope where the probe 104 is used in conjunction with a standard bronchoscope probe and delivered into the lungs or other location by the primary device. As another example, the probe 104 may be utilized with robotic navigation tools and delivered into a particular anatomical location by these tools.

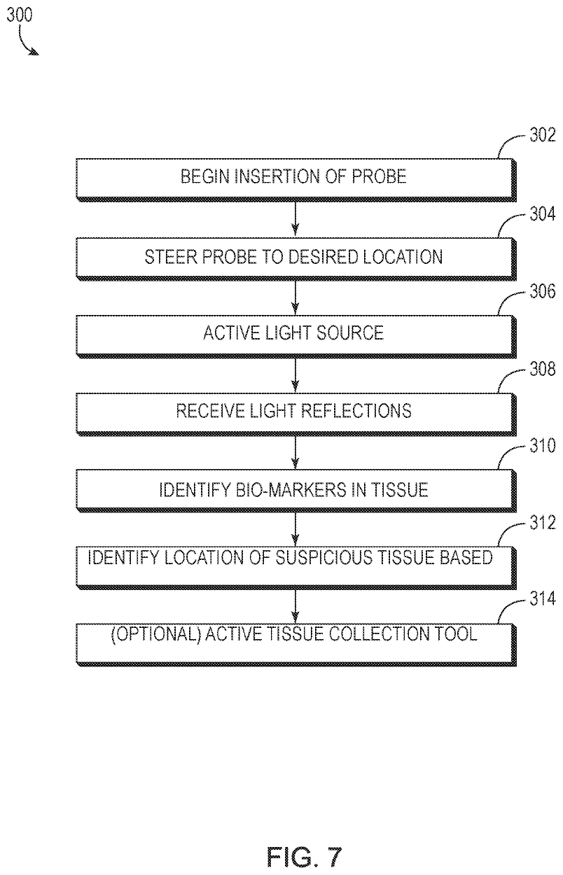

FIG. 7 illustrates a method 300 of utilizing the system 101 with or without a primary device 200, e.g., with device 100 as a standalone or integrated into another device. With reference to FIG. 7, the method 300 includes operation 302 and the probe 104 is inserted into the body. For example, the probe 104 may be inserted by being attached to a guide wire and threaded into location by the guide wire. As another example, the probe 104 may be inserted on its own with the structure of the probe 104 acting as the guide wire. The insertion may be based on a particular orifice or entry point depending on the tissue to be analyzed, e.g., via the mouth for entry into the lungs, via the rectum for entry to the colon, etc.

In operation 304, the probe 104 may be steered to a desired location. For example, the user may manipulate the handle 102 and/or the probe 104 at a location near the handle 102, to direct the probe 104 into a desired location. As another example, the user may direct the probe 104 through a working channel 202 of a primary device to steer the probe 104 to the desired location. In yet another location, the user may utilize the motor assembly 113 to drive the probe 104 to the desired location.

It should be noted that in many embodiments, the user may utilize the location sensors 114 to help direct the probe 104. For example, the user may utilize an ultrasound device to determine a location of the probe 104 and view the progress through the body. As another examples, the user may utilize an ultrasound, MRI, CT, and/or X-ray machine to capture images of the body with the probe 104 position. Also, it should be noted that position of the probe 104 may include horizontal, vertical, linear, and rotational motion (e.g., via the motor assembly 113 as discussed in more detail below). For example, it may be desirable to rotate the probe such that one or more of the sensing segments is positioned adjacent to or against a tissue area of interest.

In some examples, the probe 104 may be coupled to a mapping or positioning element, such as the motor assembly 113 or visual indicators that helps a user, such as a doctor, understand where within the body or organ the probe 104 is positioned. For example, two dimensional coordinates (such as X and Y coordinates) may be known based on the ultrasound, X-ray or other external positioning elements. The integrated positioning element, e.g., motor assembly 113, helps to identify a movement in a third plane, such as along a Z axis. As a specific example, after navigating the probe 104 to a specific area within the body, the movement of the probe 104 may be controlled, such as by the motor assembly 113, or may be visually identifiable by the user, such as visual indicators or marks, that correlate the amount of movement in a particular plane or axis. This information can be used to generate a three dimensional map for the location of the probe 104, especially as it continues to move along the selected axis. The integrated positioning element or the information determined by a positioning element can be integrated or correlated with additional image information, such as an ultrasound to X-ray, in order to generate a visual output to the user as the probe 104 is navigated within the body. In some embodiments, the 3D location may be identified with reference to a particular datum or reference point, where the datum or reference point may be an anatomical position within the body and/or an artificial or tagged location (e.g., fiducial marker added to the body or determined based on motion or navigation of the probe 104 itself within the body). As one example, after biopsy, a working channel in the probe may be used to deposit a gold or other inert pellet at the location of the analysis and/or biopsying. This may allow the location to be easily identifiable in secondary procedures and analysis.

In some examples the motor assembly 113 may enable the probe 104 to move or one or more segments of the probe 104 to move in horizontal, vertical, linear, and rotational motions. These components may allow for data to be gathered over a larger range of a sample or tissue area. The motions may be user controlled, such as through manipulations of the handle 102, or the motions may be automated based on information and data gathered during use or prior to use of the probe 104 as well as through the use of algorithms. When the motions are automated the probe 104 may capable of collecting more accurate data from samples or tissue areas at more precise intervals, such as collecting data at every 1 mm interval of linear motion or every 15, 45, 90, or 180 degrees or other desired range of rotational intervals.

In other examples the integrated positioning element may be independently capable of indicating the position of the probe 104, as well as generating a three dimensional map or visual output, through data received from components on the probe. For example, sensors, fibers, or other components may generate data indicating the position of the probe 104. The use of external or integrated positioning elements may allow the probe 104 to be operated both manually or automatically. In examples that are operated automatically, the probe 104 may collect more accurate data than is otherwise possible with manually controlled examples.

Once the probe 104 is in the desired location or as it is being directed to the desired end location but at a waypoint, the method 300 proceeds to operation 306 and the light source 110 is activated. In some embodiments, the light source 110 may be activated to generate multiple-excitation discrete wavelength pulsed sources spanning a portion of the spectrum, such as by spanning the visible spectrum, and/or may include a white-light pulsed source. The light source 110 may include an array with multiple sources, which may be substantially monochromatic sources or may be broader-band sources. As noted above, the light sources 110 may be activated via the processing elements. In some embodiments, the light source 110 may include multiple light sources that may be activated in sequence, e.g., a predetermined pattern. The sequence of the emitting lights may be used to determine characteristics of the tissue based on the reflectance, e.g., knowing that a first light at a first position is illuminated at a first time and a second light at a second position is illuminated at a second time provides spatial information to the analysis of the received first and second light reflections.

As the light source 110 is activated, light is transmitted from the light source to the transmitting fibers 118. In some examples, the light generated by the light source 110 may be collimated and/or spectrally separated before being optically transmitted to the transmitting fibers 118. For example, the light from light source 110 may be collimated by a lens (e.g., an achromatic lens that removes unwanted colors). The collimated light may be spectrally separated with a diffractive element (e.g., transmissive diffraction grating, reflect diffractive grating, prism, grism, or the like). The spectrally separated light may be focused by the lens into the transmitting fibers 118 that then transmit the light to the tissue area or sample.

The transmitting fibers 118 may then direct the light towards the tissue. In some embodiments, the probe 104 outer wall 124 may be flexed and bent against the tissue, such that the transmitting fibers 118 are adjacent or directly engaging the tissue. Additionally, because the transmitting fibers 118 extend along a length (e.g., radially or longitudinally), the transmitting fibers 118 can deliver light to a larger surface area of tissue than conventional spectroscopy devices. In some embodiments, the light source 110 may provide light to the transmitting fibers 118 in series or in another pattern. For example, in the FIG. 5A example, fibers 118 arranged closest to the handle 102 may be activated first, followed by the next closest fibers 118 and so on. Similarly, in the FIG. 4A example, the fibers 118 positioned at a first radial location may be activated first, followed by adjacent fibers 118 extending around a first direction of the circumference.

The method 303 may then proceed to operation 308 and the receiving fibers 120 receive the reflections of the delivered light. For example, the receiving fibers 120 are configured to capture and transmit the light delivered by the transmitting fibers 118 after it has interacted with the tissue. In other words, the scattered and reflected light from the tissue or other sample may be collected or received by the receiving fibers 120.

The captured or received light is then delivered by the receiving fibers 120 to the light sensors 122. In some embodiments, the captured or received light may be collimated by a lens before transmission to the light sensors 122. Additionally, a diffractive element may also be used to remove spectral separation of the light to allow the lens to better direct the light to the light sensor 122, such as light detectors (e.g., charged-coupled devices).

In embodiments where the light source 110 activates the transmitting fibers 118 at different times, the light reflections received at the different spatial locations of the receiving fibers 120 can be useful in analyzing information about the tissue. In other words, knowing a first location of a light delivery by a first transmitting fiber 118 can provide information on the tissue based on the reflections captured or received at the different locations of the receiving fibers 120.

In operation 310, the system 101 analyzes the captured light to identify cancer biomarkers in the tissue or other tissue characteristics. In one embodiment, the biomarkers may be indicative of cancer, e.g., cancer biomarkers. For example, variations in wavelength, direction, slope, absorption, and the like, can be used to determine whether the tissue is healthy, suspicious, or the like. Examples of wavelengths and analysis that may be done are described in U.S. Pat. Nos. 8,406,858 and 9,814,449 incorporated by reference herein. Examples include methods including cluster analysis, support vector machines, random forest classification, as well as neural network, deep learning networks and other artificial intelligence (AI) analyses. Specific examples are discussed in more detail below.

In operation 312, the system 101 may identify locations of suspicious and/or healthy tissue based on biomarkers and position of the probe 104. For example, as noted above in operation 304, the probe 104 location in the body may be tracked, such that areas where a suspicious tissue is identified, may be correlated to a location in the body. Further, given the correlation between probe movement and sensing locations, e.g., by moving the probe in discrete intervals and activating the light generator at those intervals, a mapping of lesions and other suspicious issues along a corridor can be determined. This allows the breadth of a particular disease or issue to be identified. To that end, in many instances, operations 304-312 may repeat multiple times as the probe 104 is slowly and precisely moved within the body.

In operation 314, the biopsy tool may be actuated to collect a tissue sample as described above. Additionally or alternatively, in operation 314, one or more treatment modalities may be activated, either with the probe 104 or with a separate tool utilized with the probe 104.

For example, the method 300 may also include treatment of the identified suspicious tissue (either at the time or in a separate procedure). As a specific example, the device 100 via the probe 104 may direct radiation via the fibers 118 towards the tissue to damage the tissue by cell death, where the light sources 110 may be varied to generate wavelengths that are determined to be sufficient to cause tissue damage. In some cases, the fibers that deliver the treatment radiation may be different from those used to analyze the tissue, but in other embodiments they may be similar. Other types of treatments, such as chemical applications or the like may be delivered via the probe and the working channel within the probe. As yet another example, the probe 104 may be configured to deliver non-thermal plasma to induce immunogenic cell death. For example, a dielectric barrier discharge plasma system may be incorporated with the probe 104 to generate plasma and cause death of the tissue cells. The fibers or other delivery wires can be used to apply voltage to the cells, where the voltage may be modulated or varied over time (e.g., pulse width modulated). In these examples, a treatment wire or fiber, such as an electrically conductive wire may be threaded through the working channel or may be integrated with the probe 104 and be configured to apply the desired treatment, such as a desired voltage, to the tissue.

As briefly noted above, in some embodiments either before operation 314 or where operation 314 is not included, the method 300 may repeat operations 304-312. For example, the motor assembly 113 may selectively steer the probe 104 and the probe 104 may activate the light sources to receive light data at predefined or otherwise known intervals within the body. Such steering and known data capture allows the probe 104 to be used to determine a 3D map or other 3D representation of tissue characteristics. Such 3D information has not been conventionally detected in spectroscopy devices, which makes localization of lesions or other suspicious tissue difficult to determine. It should be noted that the motor assembly 113 may assist in generating the 3D information by moving the probe 104 at predefined and precise intervals. In other embodiments, a manual guide assembly, e.g., precise locators or markers, may be positioned on the handle that visually indicate the movement of the probe 104 within the body. For example, as the user navigates the probe 104, the user can use the visual markers to determine when to activate the light sources/light collection and generate known intervals and locations to generate the 3D mapping. The various implementations and embodiments may be used to generate a 3D column or other shaped mapping of tissue as being suspicious or not suspicious, which allows precise location of, for example, cancer and other disease.

Further, in many instances, lesions or other suspicious tissue may be not be in easily accessible areas. For example, in the lungs, lesions are often located away from the bronchial pathway, and the probe 104 will need to be navigated through bronchial pathway tissue and into the SPN. Relatedly, while the probe 104 may include an integrated biopsying element, in other embodiments, the biopsying elements 150 may be omitted and the probe 104 may be used solely for diagnosis or detection of tissue characteristic.

In various examples, the system 100 and probe 104 may utilize electromagnetic properties to characterize tissue, with specific embodiments using certain spectroscopic properties illustrative of morphological and biochemical information to characterize tissue. Fluorescence is measured from samples (e.g., as collected by fibers 130) or tissue areas and may result from intrinsic or extrinsic fluorescence. “Intrinsic fluorescence” refers to auto fluorescence spectra (AFS) from molecules that occur naturally in a sample or tissue area while “extrinsic fluorescence” refers to fluorescence from molecules that have been added to the sample by an external mechanism.

Optical spectroscopy, such as method 300, provides a methodology to diagnose disease by quantitatively evaluating changes in tissue morphology and composition. Light interacts with biological tissue in a variety of ways. Various types of tissue fluoresce, absorb, and scatter light in different regions of the electromagnetic spectrum and by different amounts. The optical properties of tissues are determined by their molecular composition and cellular morphology. Optical techniques utilize accurate measurements of these absorbed or scattered signals to identify benign versus malignant tissues. Based on this principle, optical diagnostic techniques have been used to identify various types of precancerous lesions and carcinomas in real-time.

However, optical properties of different organs are very different. Optical spectroscopic features may need to be established for individual tissue types to provide identification of benign versus premalignant/malignant tissue. A large majority of scattering events in biological tissue are elastic. Elastic scattering spectra (ESS) and diffuse reflectance spectra (DRS) primarily probes morphological features and has proven to be sensitive to histopathologic grade of cancer in different organs. ESS mainly consist of Rayleigh scattering composed with single scattering events. DRS consist of both the single and multiple scattering events and composition depends on the separation between source and detector fibers.

ASF may be combined with ESS and DRS to extend the classification for benign versus premalignant/malignant tissue. While fluorescence is a weaker process than scattering, it potentially allows for identification of specific endogenous fluorophores whose concentrations may vary with disease state. Analysis of both types of observed signals is therefore desirable. Such analysis may involve estimation of tissue scattering and fluorescence properties, solution of the scattering problem, and prediction of the effect of scattering and absorption on fluorescence signals.

The system 100 and probe 104 may be used to collect data for analysis via method 300 or using other optical spectroscopy techniques. Similarly, once the data is collected it can be analyzed in a variety of different manners.

One example method of data analysis includes an assessment of Excitation Emission Matrix (EEM). An EEM is a 3D matrix with each matrix element representing an excitation wavelength, an emission wavelength, and an intensity. If sᵢ(λₛ) is the ith intensity source spectrum (for i=1, 2, . . . , m) and rᵢ(λᵣ) is the intensity spectral response of the tissue due to the ith source (for i=1, 2, . . . , n), then the function of input wavelength λₛ (excitation wavelength) and output wavelength λᵣ (received wavelength) that may be determined from the radiation collected through the electromagnetic subsystem at the front end of the receiver (e.g., receiving fibers) is

[symbol]

(1)
where the value of i takes on values from 1 to m, the number of sources. A matrix of values may be formed from this data object by having the receiver perform integrations. The total source power levels for the light sources are determined from the integrals.

[symbol]

(2)
where the integration interval includes all values at which the source emits power. The emission linewidth of the ith source is denoted Δλ_{z}, with the integral being performed over this linewidth. The receiver power levels are determined from integrals over intervals λⱼ^{±} such that the ijth component of the m×n matrix M is given by

[symbol]

(3)
where optimal values of may be determined by the experimenter and/or data processor. The source may also be parsed as the output by writing that

[symbol]

(4)

Evidently,

[symbol]

(5)

When a spectrometer is used within the receiver, the intervals Δλⱼ=λⱼ−λⱼ can be very fine, for example, as fine as 0.01 nm although spacings of 0.4 nm are also in use, but this example is not intended to be limiting since larger intervals may be used as well. In practice, decisions on tissue characterization may be made on some total number of features N_{f} that is generally less than 15. In an exemplary system with eight sources, then, at most two output points per source may be of interest. But further processing may advantageously use many more points for stability and identification in the processing algorithm before the task of reducing the EEM to features is performed.

A square matrix may be generated from the n×m matrix to easily define the elastically scattered terms with the matrix diagonal. A condition on the spectral widths may be exploited to do this: When Δλ₁≥Δλⱼ for all values of i and the values nⱼ≈Δλ₂/Δλⱼ are approximated as integers, then

[symbol]

(6)
to define an nₘ×nₘ square matrix whose diagonal is nonzero but may include some off-diagonal filling. The matrix M is then considered as square with diagonal elements representing a measure of the scattering and absorption characteristics (elastic scattering elements) of the tissue sample weighted by the spectral strength of the source in that region. Off-diagonal elements of the matrix represent a measure of higher-order nonlinear processes such as fluorescence. The diagonal is defined by the points that were generated where the λⱼ interval covers the source interval.

Next, the nm x nm matrix may be expanded to generate an n_{c}×nₘ×nₘ space. An algorithm is applied to the diagonal elements to separate them into n_{c} components corresponding to various measures of scattering and absorption, while the off-diagonal elements are expanded using the identity operation. Notably, the expansion of the matrix is a completely invertible operation and none of the original information is lost.

The expansion of the diagonal elements into three components may be accomplished by first generating the diffuse reflectance vector D(X) from the diagonal elements of the nₘ×nₘ square matrix M. The diffuse reflectance corresponds to the elastic scattering. The vector may be generated by performing the following operation:

[symbol]

(7)

The generation of the nₘ×nₘ square matrix with nonzero diagonal elements results from the Sᵢⱼ used in the above equation being nonzero. In a particular embodiment, a number of operations are performed, including reduction of single scattering and separation of the remaining diagonal elements into those that come from absorption and those that are due to multiple scattering.

The oscillations may be split from the diffuse reflectance by subtracting the windowed average of the diffuse reflectance from the diffuse reflectance:

$${S}_{i} = {D}_{i} ( λ ) - {\sum }_{j} {W}_{ij} {D}_{j} , \qquad ( 8 )$$

where Wᵢⱼ is a window function of width w. From the windowed diffuse reflectance, the remaining two elements may be extracted. This may be performed by noting the following unique features of turbid media such as tissue and absorption spectra. The diffuse reflectance due to multiple scattering contributions from particles smaller than a wavelength is a monotonically decreasing function of wavelength and for the visible region of the electromagnetic spectrum is approximately linear for scattering elements Δna<0.2 μm, where Δn is taken to be the difference in the index of refraction between the nucleus and the cytoplasm in a cell, which is approximately 0.1. The second element aiding in the separation is the broadening of absorption features for elements in tissue, which generates an imbalance in the first derivative (divided difference) of the smoothed diffuse reflectance that can be averaged out of the first derivative (divided difference) if the entire absorption feature is captured or can be balanced out of the response using centroids to balance the first derivative.

The monotonically decreasing function may then be extracted from the balanced first derivative (divided difference) and subtracted from the smooth diffuse reflectance, yielding the absorption contribution of the tissue response. The monotonically decreasing function and the absorption contribution are the second and third elements of the diagonal expansion.

The new 3×nₘ×nₘ space generated by the expansion of the matrix characterizing the tissue may be reduced to a number of features that are independent to enable classification of the signature from this array of digitally filtered input data. There are numerous automated techniques known to those of skill in the art for estimating the number of terms that could be used for attempting to recognize a data pattern. When too many factors are used, the recognition becomes non-unique; when too few are used, the recognition cannot be made. In embodiments of the invention, the spectral broadening in the tissue may place a limit on the minimum meaningful spectral width of a feature to be used. The diagonal of the square matrix may then be reduced to factors by summing over diagonal elements, whereas as a whole off-diagonal elements can be used in a similar manner.

A common term for methods that find the number of independent features from each data sample is “principal component analysis.” When many tissue samples are lumped together such that there are Nₛₐₘₚₗₑₛ×n_{c}×n×m arrays processed simultaneously, the techniques are often called “multispace techniques.” Here, single-data-point techniques are those that are used. Once the data have been reduced to N_{f} independent values such that the problems may be considered to be an identification problem in an N_{f}-dimensional space, permitting any of several pattern-classification techniques known to those of skill in the art to be implemented by the pattern classifier to classify the result into one of Nₚₐₜₜₑᵣₙₛ. The pattern-classification techniques may be used generally to separate the N_{f}-dimensional space by (N_{f}−1)-dimensional hyperplanes and find in which box the data point resides.

During use, data from a sample (e.g., tissue) are collected by irradiating the sample with light and receiving light scattered from the sample. Multiple distinct excitations of the sample are identified from the received light, which permits on-diagonal components of the EEM corresponding to ESS and DRS and off-diagonal components corresponding to AFS of the EEM to be identified simultaneously. The EEM is generated from the identified components.

Once the EEM has been generated, it can be used to derive a plurality of spectral measures, which in one embodiment include absorption, fluorescence, multiple scattering, and single scattering. The group of measures is used for each point within a spatial distribution to generate a contour plot.

The spectral measures may then be used to classify the sample. In one embodiment where the sample comprises biological tissue, this is performed with a cluster analysis that separates the tissue into three categories—“normal,” “precancerous,” and “cancerous.” Such cluster analysis may group together items using a statistical analysis of a Euclidean distance measure in three-space generated with the fluorescence, scattering, and absorption measures.

Other methods of data analysis may include principal component, artificial neural networks (ANN), and various statistical analyses that may be employed to develop a preliminary classification scheme for in vivo diagnosis of tissue. For example, classification of AFS, ESS, and DRS may be performed in a binary classification scheme using an ANN. Other alternatives include the use of logistic regression, Gaussian processes, support vector machine, random forest, and boosted tree regression algorithms to improve the classification scheme.

It should be noted that the probe 104 embodiments discussed herein are meant to be illustrative only. The probe 104 may be coupled or inserted through a working channel in a variety of devices, including multiple types of scoping devices. Due to the flexibility of the probe and the multiple sensing segments positioned at locations in addition to or separate from a terminal end of the probe 104, the probe 104 can be used in multiple locations within the body.

The description of certain embodiments included herein is merely exemplary in nature and is in no way intended to limit the scope of the disclosure or its applications or uses. In the included detailed description of embodiments of the present systems and methods, reference is made to the accompanying drawings which form a part hereof, and which are shown by way of illustration specific to embodiments in which the described systems and methods may be practiced. These embodiments are described in sufficient detail to enable those skilled in the art to practice presently disclosed systems and methods, and it is to be understood that other embodiments may be utilized, and that structural and logical changes may be made without departing from the spirit and scope of the disclosure. Moreover, for the purpose of clarity, detailed descriptions of certain features will not be discussed when they would be apparent to those with skill in the art so as not to obscure the description of embodiments of the disclosure. The included detailed description is therefore not to be taken in a limiting sense, and the scope of the disclosure is defined only by the appended claims.

From the foregoing it will be appreciated that, although specific embodiments of the invention have been described herein for purposes of illustration, various modifications may be made without deviating from the spirit and scope of the disclosure.

The particulars shown herein are by way of example and for purposes of illustrative discussion of the preferred embodiments of the present disclosure only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the disclosure, the description taken with the drawings and/or examples making apparent to those skilled in the art how the several forms of the disclosure may be embodied in practice.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

Of course, it is to be appreciated that any one of the examples, embodiments or processes described herein may be combined with one or more other examples, embodiments and/or processes or be separated and/or performed amongst separate devices or device portions in accordance with the present systems, devices and methods.

Finally, the above discussion is intended to be merely illustrative of the present system and should not be construed as limiting the appended claims to any particular embodiment or group of embodiments. Thus, while the present system has been described in particular detail with reference to exemplary embodiments, it should also be appreciated that numerous modifications and alternative embodiments may be devised by those having ordinary skill in the art without departing from the broader and intended spirit and scope of the present system as set forth in the claims that follow. Accordingly, the specification and drawings are to be regarded in an illustrative manner and are not intended to limit the scope of the appended claims.

What is claimed is:

1. A device comprising:
a light generator; and
a probe in optical communication with the light generator, comprising:
a flexible outer wall having a first end and a second end opposite the first end;
a plurality of receiver fibers having ends that are arranged to extend along a portion of a length of the outer wall and are positioned between the first end and the second end;
a plurality of transmitter fibers having ends that are arranged to extend along a length of the outer wall, adjacent to the plurality of receiver fibers.

2. The device of claim 1, wherein the plurality of receiver fibers are positioned directly adjacent to the plurality of transmitter fibers and the portion of the length of the outer wall is optically transmissive.

3. The device of claim 1, wherein:
the ends of the plurality of receiver fibers extend longitudinally along the length of the outer wall; and
the ends of the plurality of transmitter fibers extend longitudinally along the length of the outer wall.

4. The device of claim 1, wherein:
the ends of the plurality of receiver fibers extend annularly around the outer wall; and
the ends of the plurality of transmitter fibers extend annularly around the outer wall.

5. The device of claim 1, wherein the plurality of receiver fibers and the plurality of transmitter fibers terminate before a distal end of the outer wall.

6. The device of claim 1, wherein the housing further comprises a channel configured to receive a tool therein.

7. The device of claim 6, wherein the tool is a tissue collection tool.

8. The device of claim 1, wherein the probe is configured to be received within a working channel of a medical device.

9. A device comprising:
a light source; and
a probe in optical communication with the light source, the probe comprising:
a body; and
a first sensing segment positioned at a first location of the body; and
a second sensing segment positioned at a second location of the body, wherein the first location and the second location are located apart from a terminal end of the probe.

10. The device of claim 9, wherein the first sensing segment is defined by a plurality of optical fibers having fiber ends oriented perpendicular to a longitudinal axis of the body.

11. The device of claim 10, wherein the second sensing segment is defined by a plurality of optical fibers having fiber ends oriented perpendicular to a longitudinal axis of the body.

12. The device of claim 9, wherein the first sensing segment is defined as a ring extending annular along an outer surface of the body and the second sensing segment is defined as a ring extending annularly along the outer surface of the body and spaced apart from the first sensing segment.

13. The device of claim 9, wherein the first sensing segment is defined as a longitudinal portion extending parallel to a portion of a longitudinal axis of the body and the second sensing segment is defined as a longitudinal portion extending parallel to a portion of the longitudinal axis of the body.

14. A device comprising:
a probe, the probe comprising:
a flexible body defining a channel;
a motor assembly coupled to the body and configured to move the body in precise movement intervals;
a light source coupled to the probe;
a plurality of fibers positioned at least partially within the channel of the body, the plurality of fibers comprising:
one or more transmitting fibers, the transmitting fibers configured to transmit light from the light source to one or more in vivo locations within a body;
one or more receiving fibers, the receiving fibers extending through the body and configured to receive light from the one or more in vivo locations, wherein the plurality of fibers are arranged to transmit and receive light through a sidewall of the flexible body; and
a light sensor coupled to the probe, wherein the light sensor translates light received from the one or more receiving fibers into digital data.

15. The device of claim 14, wherein the one or more fibers are positioned so that they emit and receive light perpendicular to a direction of a length of the outer wall.

16. The device of claim 14, wherein ends of the one or more fibers are positioned in one or more sensing segments along the outer wall, the sensing segments including fibers in one or more arrangements.

17. The device of claim 14, the device further comprising:
one or more processing elements, the processing elements configured to control the motor assembly and analyzing the digital data to determine tissue characteristics at the in vivo location.

18. The device of claim 14, wherein a portion of the sidewall is transparent or includes an aperture to allow the plurality of fibers to transmit and receive light therethrough.

19. The device of claim 14, wherein the body includes a first end comprising a deployable cutting element.

20. The device of claim 14, the device further comprising:
a navigational component, the navigational component configured to determine the position of one or more portions of the probe or a component of the device by utilizing location data generated from one or both of the probe or external monitoring devices.
